(19) United States
(12) Patent Application Publication
(10) Pub. No.: US 2010/0318086 A1
(43) Pub. Date: Dec. 16, 2010

(54) METHOD AND APPARATUS FOR TREATING PERIPROSTHETIC FRACTURES OF THE DISTAL FEMUR

(76) Inventor: Mitchell Winemaker, Ontario (CA)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 12/867,625

(22) PCT Filed: Feb. 5, 2009
(86) PCT No.: PCT/CA2009/000139

§ 371 Date: Aug. 13, 2010

Related U.S. Application Data
(60) Provisional application No. 61/029,331, filed on Feb. 16, 2008.

(51) Int. Cl. A61B 17/80

(52) U.S. Cl. 606/70

(57) ABSTRACT

A medical device is provided for attachment to a patient's femur to treat a fracture in the distal region of the femur, in a patient with an artificial knee joint. The apparatus includes first and second elongate plates for attaching to the femur at the location of the fracture. The plates include coupling means to rigidly coupling the plates to the femoral component of the artificial knee joint. The apparatus further includes a transverse link for connecting the first and second plates together in a spaced apart relationship to form a rigid linkage between said plates. The invention further includes a system for treating a fracture of the femur, especially in patients with weak bones, by attaching such plates in an opposed spaced apart relationship on the lateral and medial sides of the femur, and fastening the lower ends of the plates to the femoral component of the knee. The transverse link is engaged to form a monolithic, rigid structure for bracing the femur.

### FIELD OF THE INVENTION

The invention relates to medical devices, in particular an implant for treating fractures of the femur. The invention further relates to methods of treating such fractures, and uses of such devices to treat femoral fractures.

### BACKGROUND OF THE INVENTION

Periprosthetic fractures of the distal femur are the most common fracture location around the knee with an incidence ranging from 0.3-2.5%. They account for more than 10,000 fractures per year in the US alone. Current fixation of these fractures has a high complication rate due to a paucity of bone remaining in the distal fragment for rigid fixation, particularly in osteoporotic bone. Using current techniques, fracture fixation is often inadequate and the fracture often heals in a malposition causing abnormal stresses on the preexisting knee prosthesis. This results in persistent pain, stiffness and potential for early failure of the total knee prosthesis. Furthermore, abandoning fracture fixation in favour of revision arthroplasty using more constrained hinge devices leads to high complications and early failure as well as more severe reconstructive problems in the future. Non-surgical treatment of these fractures is rarely an option because of the medical complications of prolonged immobilization and surgical fixation is generally accepted as the standard of care in most instances.

It has been proposed to stabilize the fractured femur by fastening a rigid component to the femur. For example, U.S. Patent Application 2004/0122430 (Hansson) discloses an implant for fixation of femoral fractures. This device, as well as others within the prior art, requires modification of the femoral implant of the artificial knee in order to anchor the rigid component. Conventional surgical techniques also include the use of an intramedulallary nail, the “Less Invasive Stabilization System” (LISS) and lateral condylar locking plate, the dynamic condylar screw and plate system, an angled blade plate, and numerous other options. However, the use of such systems still appears to result in high complication rates, especially in osteoporotic patients.

It has also been proposed to provide a bone plate for fixing a femur, which couples with the femoral component of an artificial knee joint. Examples of such systems are described in U.S. Pat. No. 5,522,902 (Yuan et al.) and published Application U.S. 2007/0225819 A1 (Eva). It is believed that these devices do not adequately address the need to rigidly fix the femur in position, with a system that is relatively straightforward to implement and can treat patients with arthritis and other conditions of weak bones.

### SUMMARY OF THE INVENTION

In light of shortcomings in the prior art, including the shortcomings noted above, it is desirable to provide an improved implant for femoral fractures, and in particular an implant that is particularly suitable for osteoporotic patients who have an existing knee prosthetic.

One problem that arises in patients with osteoporosis is a lack of sufficiently dense bone at the distal end of the femur to anchor a conventional plate or other component for supporting the fractured femur. The present invention addresses this problem by utilizing an existing artificial knee in the patient to anchor a plate which supports and immobilizes the fractured femur.

The invention relates generally to a method and apparatus to treat a fracture of the distal femur in a patient with weakened bones, by rigidly anchoring a pair of opposing plates to the femoral component of an artificial knee. The plates are fastened to the distal end of the patient's femur to allow bony healing and improve overall alignment of the knee prosthesis with respect to the limb. Most femoral components of total knee replacements have small metal slots on the distal-medial and distal-lateral edges of the prosthesis that are used for attachment of an insertion handle when implanting the device. These slots are not part of the articulating surface of the implant and may be as such used to anchor a fracture fixation device to improve the overall rigidity and alignment of the fracture and thus the limb. These slots typically serve no function after implantation of the artificial knee, and as such they are available to serve as anchoring points for other components.

According to one aspect, the present invention comprises a pair of elongate plates, consisting of medial and lateral condylar plates, which are pre-contoured or which may be contoured on a customized basis prior to use, for engaging opposing (lateral and medial) sides of the distal end of the femur. The plates include attachment means for attaching the plates to the femur, such that at least the lateral plate overlaps the fracture for fixation thereof. At their distal ends, the opposing plates each include an engagement means for anchoring the plates to the femoral component of an artificial knee prosthesis which has been previously attached to the patient's femur. The engagement means may comprise any suitable means to rigidly attach the plates to the femoral component, as described below, so as to rigidly anchor the distal end of each plate to the artificial knee component. Preferably, the engagement means makes use of an existing feature of the femoral component and does not require modification of the femoral component.

In one version, the engagement means which anchor the medial and lateral condylar plates to the femoral component of the knee prosthetic with the engagement means comprise a rectangular metal prong or tab that fits into the pre-existing anchoring slot on the femoral prosthesis.

The lateral plate extends proximal to the fracture to assist reduction, alignment restoration and fixation. The shorter medial plate primarily serves as a link with the lateral plate to allow compression of the plates to increase the strength of the fixation to the femoral prosthesis. The medial plate is short in relation to the lateral plate, so as to extend only a minimal distance along the femur to minimize the possibility of disruption to the blood supply. The medial plate need not extend along the femur to the same extent as the lateral plate, since it is contemplated that the primary function of this plate is to provide structural support to the lateral plate, rather than providing substantial anchoring or support of the femur itself.

According to another aspect, at least one rigid transverse link joins the first and second plates adjacent to their distal ends, so as to mechanically link the plates together to form a substantially rigid structure. In one embodiment, the structure has a generally H-shaped configuration when attached to a patient. The link holds the plates in place on the opposing lateral and medial sides of the femur in a vertical orientation. The link may consist of one or more fixed angle compression screws, or other suitable rigid member which rigidly joins the respective plates together. The plates and transverse link when fastened together form a rigid monolithic structure that resists movement relative to the femoral component, and as such this structure provides a rigid support for the fractured femur.

The transverse link may comprise a compression bolt spanning the respective plates. A nut on the end of the bolt may be tightened to place the link under tension and thereby compress the plates together. The plates may be provided with openings aligned with each other when the plates are engaged to the artificial knee component, which receive the bolt. The link connects the two plates at a fixed angle. The amount of compression provided by the link is sufficient to compress the two distal tabs into the slots in the implant with sufficient force to rigidly lock the plates into position. The link passes through an opening which is formed within the patent's femur bone and is positioned partway between the anterior and posterior aspects of the femoral implant. Preferably, this position is approximately closer to the anterior one-third and posterior two-third junction.

Several of the components described herein are known, or may be adapted from known medical device components. Precontoured plates that fit over the medial and lateral condyles of the distal femur are known to the art, as are non-contoured plates that may be contoured on site to fit the patient. Fixed angled devices are generally known for linking bone screws at a fixed angle to a plate.

According to another aspect, the invention relates to a method of treating fractures located at or near the distal end of the femur, in patients with an existing artificial knee. According to this aspect, components as described above are utilized. The respective plates may be supplied either in a pre-contoured form or in a non-contoured form, and are contoured to fit and engage the patient's lower femur. The method involves the surgical attachment of the respective plates on opposing sides of the distal femur, anchoring the plates to the femoral component of the artificial knee, and linking the plates together with the transverse link. The plates may be held in position by a temporary clamp prior to their attachments as described above. The invention preserves the integrity of the knee prosthesis and the collateral ligaments of the knee, and the plates can be removed if necessary once the fracture has healed.

The present invention will now be described by way of a detailed, non-limiting description of embodiments of the present invention. It will be understood that the detailed embodiments described herein are not intended to limit the scope of the invention, but are merely intended to illustrate the present invention.

### BRIEF DESCRIPTION OF THE DRAWINGS

FIG. 1 is a side elevational view of a portion of an apparatus according to the present invention;

FIG. 2 is a side elevational view, opposed to FIG. 1, of another side portion of the apparatus;

FIG. 3 is a perspective view of a temporary link for retaining the plates in position during surgical installation thereof;

FIG. 4 is a front (anteroposterior) view of the present invention, fixed to a patient with a distal femur fracture;

FIG. 5 is a perspective view of the first embodiment of the invention, engaged to a fractured femur;

FIG. 6 is a further perspective view as in FIG. 5, showing an opposing side thereof;

FIG. 7 is a view as in FIG. 4, of an alternative embodiment of the invention;

FIG. 8 is an exploded view of components of the embodiment of FIG. 7;

FIG. 9 is a perspective view of a further alternative embodiment of the invention;

FIG. 10 is a perspective view of a still further embodiment of the invention;

FIG. 11 is a perspective view of the transverse link portion of the embodiment shown in FIG. 10.

### DETAILED DESCRIPTION

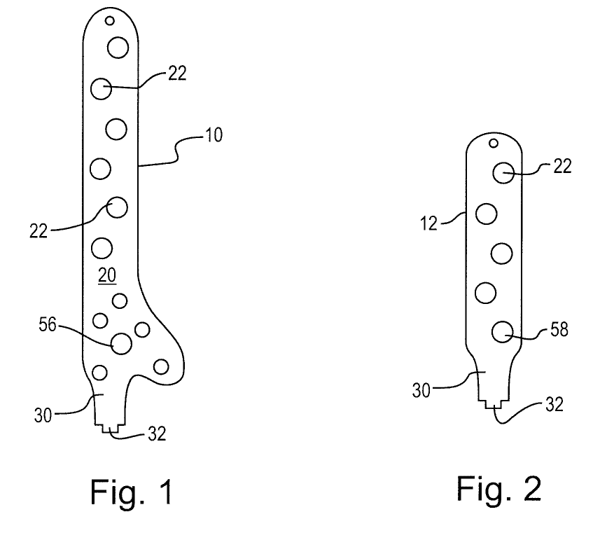

Turning to FIGS. 1-6, a first embodiment of the invention includes a pair of elongate plates 10 and 12 that are configured to be surgically attached to opposing sides of a patient's femur 14, to immobilize the femur in the region of a distal fracture. The apparatus is intended for use for a patient who has an existing artificial knee, which includes a femoral component 16 fixed to the distal end of the femur. The apparatus is configured to engage this femoral component. Most conventional femoral components include opposed lateral slots 34, which are provided to engage an insertion tool when implanting the device, and have no intrinsic function after implantation of the knee joint. The first embodiment described herein is configured to engage such slots 34.

The first of the plates comprises a lateral plate 10, seen in FIG. 1, comprising an elongate plate body 20 contoured to contact the distal lateral region of the femur. The plate body is provided with a plurality of openings 22 to receive screws or nails 24 for attaching the plate to the femur 14 across the location of the fracture 26. The plate 10 may be supplied in a pre-contoured form, or alternatively as a substantially flat plate which may be contoured prior to implantation to conform to the patient's femur, similar to a conventional distal lateral femoral plate. Typically, such a plate is cast from surgical-grade stainless steel, and may be sufficiently malleable to permit shaping of the plate prior to implantation. Extending distally from the base of the plate body is a generally rectangular protrusion 30 which is substantially co-planar with the plate body. The protrusion 30 is configured to extend downwardly alongside the lateral side of the femoral component 16 of the artificial knee, to a position adjacent to the distal end of the femoral component. The distal end of the protrusion comprises an engagement means to anchor the plate to the femoral component in a relatively rigid fashion. As seen in FIGS. 1, 2 and 4, the engagement means comprises an inwardly-facing rectangular tab 32, configured to fit snugly within the pre-existing anchoring slots 34 within the femoral component 16 of the artificial knee. Tabs 32 should be configured to avoid disruption or impingement on the medial and lateral collateral ligaments which stabilize the femur to the tibia throughout motion. To do so, the shape of the tabs should curve underneath the collateral ligaments from a position just anterior to the midline such as the anterior one-third, posterior two-thirds junction for example. According to one embodiment, tabs 32 comprise a material that permits customized shaping to protect the patient from any injury to these ligaments.

In order to maintain proper alignment of the plates, it is preferred to provide the plates in a precontoured form, such that only minimal adjustment to the plates, or no adjustment, is required to maintain proper alignment. Medial plate 12 may be somewhat contoured in situ, but it is preferred for lateral plate 10 to be supplied in essentially a precontoured form with only the distal tab portion 32 being somewhat contoured in situ.

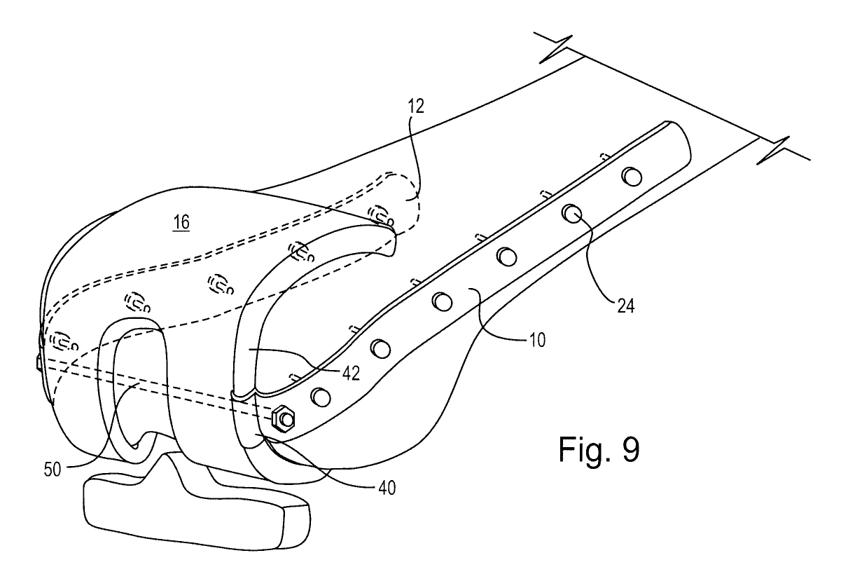

It will be seen that a variety of means may be used to rigidly anchor plates 10 and 12 to the femoral component. One such anchoring means that may be adapted for the present invention is described in U.S. Patent Application 2007/0225819, incorporated herein by reference. A further alternative anchoring means is shown in FIG. 9, which comprises a hollow semi-cylinder 40 configured to wrap around an edge portion 42 of the femoral prosthesis 16. This latter engagement means may be shaped on site, to snugly and firmly engage the prosthesis. An engagement means of this type would be useful if the femoral component does not have conventional slots on the sides thereof.

The second plate 12 comprises a medial plate, which engages the distal medial side of the femur. The medial plate is shorter in length than the lateral plate, in order to not interfere with the blood supply on the medial side of the femur. Apart from the shorter length, the medial plate is generally the mirror image of the lateral plate.

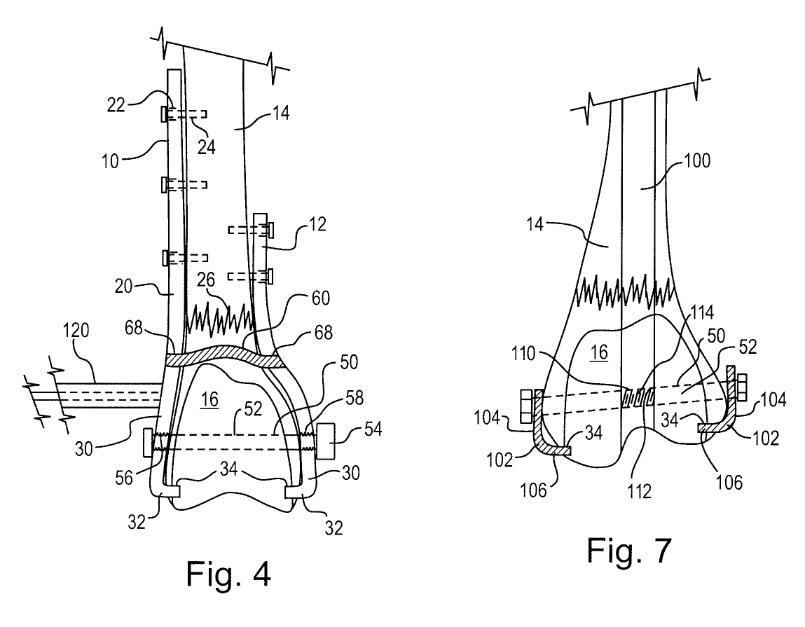

The lateral and medial plates are rigidly connected together by means of a rigid transverse link 50. Preferably, the link urges the respective plates together to compress the femoral implant between the plates so as to form a mechanically rigid monolithic structure consisting of the opposing plates 10 and 12 and the transverse link 50. This rigid structure is believed to be more robust and to provide more support to the femur than a single plate attached to the femur, and is of particular value if the patient's bones are osteoporotic or otherwise weaker than normal. In order to direct all or most of the compressive forces through the artificial knee in order to provide the least stress on the femur, the link is located relatively close to the distal ends of the plates, as close as reasonably possible to the anchoring members. As particularly seen in FIG. 4, the link 50 comprises a shaft 52 extending between the respective plates 10 and 12. The shaft 52 preferably comprises a fixed angle threaded compression screw, which is retained by a compression nut 54.

Plates 10 and 12 include holes 56 and 58 which are aligned relative to the distal ends of the plates to allow targeting of the threaded shaft 52 to cross both corresponding holes in the plates 10 and 12. The lateral plate hole 56 is threaded so the screw will lock into the plate at a fixed angle and the medial plate 12 is unlocked with a slightly larger hole 58 to improve accuracy of screw placement. Compression is applied by attachment of threaded nut 54 to the medial side that can be tightened with a ratcheted torque wrench. The initial drill hole for screw placement is made through a threaded drill guide that fits in the lateral plate holes 22.

Link 50 is assembled onto plates 10 and 12 in medical procedure which involves forming an opening through the distal end of the patient's femur bone, aligned with holes 56 and 58. At this location, the patient's femur bone is retained within the femoral component of the artificial knee. Shaft 52 is inserted through this opening, and one end of shaft 52 is threaded into threaded opening 56 of the lateral plate. The opposing end of shaft 52 protrudes through opening 58, and threaded nut is fastened to this protruding portion.

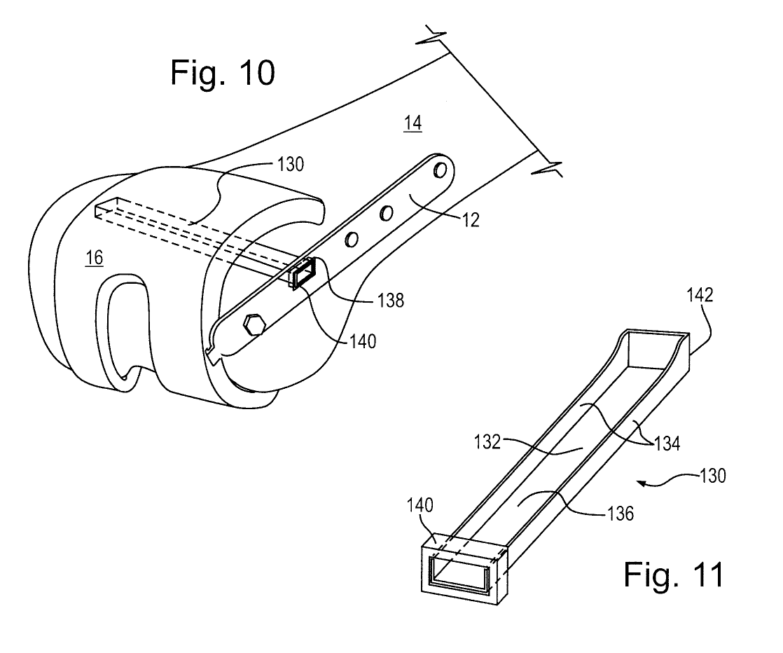

FIGS. 10 and 11 illustrate an alternative embodiment of the rigid link. In this version, the rigid link comprises a blade plate 130, consisting of an elongate box-shaped structure having a flat base 132, vertical sidewalls 134, and an open top 136. The profile of plate 130 is thus generally U-shaped with a flat bottom and vertical sides. Plate 130 fits within opposes openings 138 within plates 10 and 12. Openings 138 are aligned when plate 10 and 12 are fastened to the patient's femur. One end of plate 130 includes an elevated rear wall 142 which engages plate 130 within one of the openings 138 within one of plates 10 or 12. Plate 130 is fastened to the other of plates 10 and 12 by means of a slide-on fastener 140. In order to install plate 130, a hole is cut within the patient's femur in a fashion similar to the embodiments of FIGS. 1-9, followed by inserting plate 130 through this opening until rear wall 142 engages one of plates 10 or 12 (depending on whether plate 130 is inserted from the medial or lateral sides). Plate 130 is then fastened to the other of plates 10 or 12 by engaging fastener 140.

Plates 10 and 12 are configured so that the linking with the prosthesis will place the femoral component 16 in about a five degree valgus angle in the coronal plane and parallel to the femur in the sagittal plane. Rotation is determined by fracture reduction and by the flexion-extension axis of the knee.

Transverse link 50 may be removed after the femur has healed to a sufficient degree, by using a strong wire cutting type instrument (not shown) to break through the link. The instrument may be inserted through one or more percutaneous small incisions. The link must be sufficiently robust to avoid breakage before bone healing and still weak enough that an instrument could be introduced to sever the link or otherwise separate the link from the paired plates.

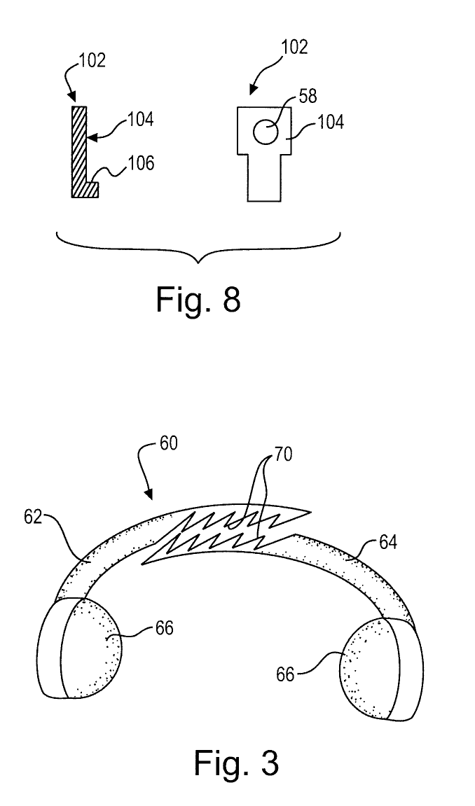

Plates 10 and 12 can be surgically implanted in patient by means of a removable coupler that holds the plates in position until they are attached to the patient's femur and/or until the plates are rigidly coupled together with the link. The coupler may comprise a conventional Weber™ clamp that holds the plates in position during the implantation procedure. Alternatively, the coupler may comprise a clamp 60 specifically configured to engage the plates, as seen in FIG. 3. The clamp comprises opposing elongate clamp jaws 62, 64 that are contoured to extend around the plates 10 and 12 when initially engaged to the femur. The ends of the clamp jaws comprise opposing protrusions 66 that engage aligned depressions or openings 68 within the respective plates 10 and 12. The opposing jaws 62, 64 comprise angled teeth 70 which engage each other in a ratcheting fashion. The respective jaws are operated by handles (not shown) which are pivotally at their upper ends, for operation by the user. The clamp provisionally links the medial and lateral plates 10 and 12 by applying a medial and lateral force to hold the plates 10, 12 to the femoral prosthesis prior to the application of the rigid link.

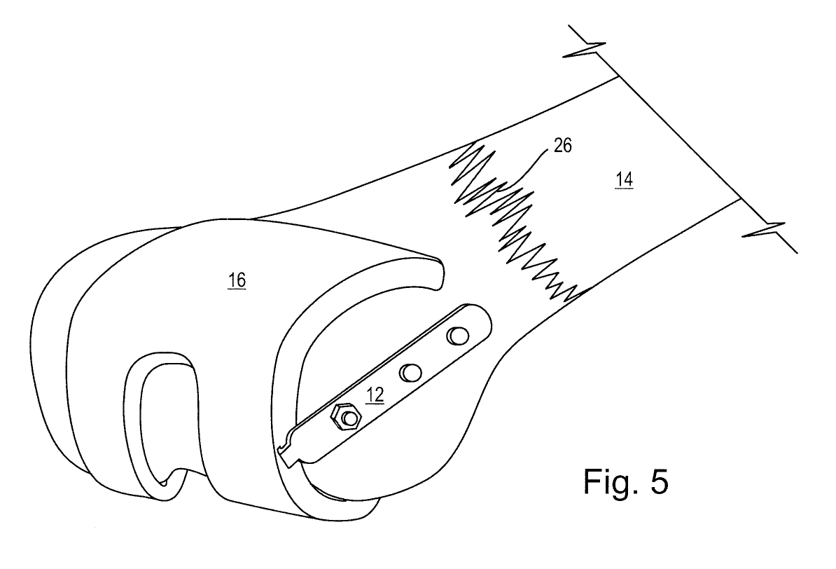

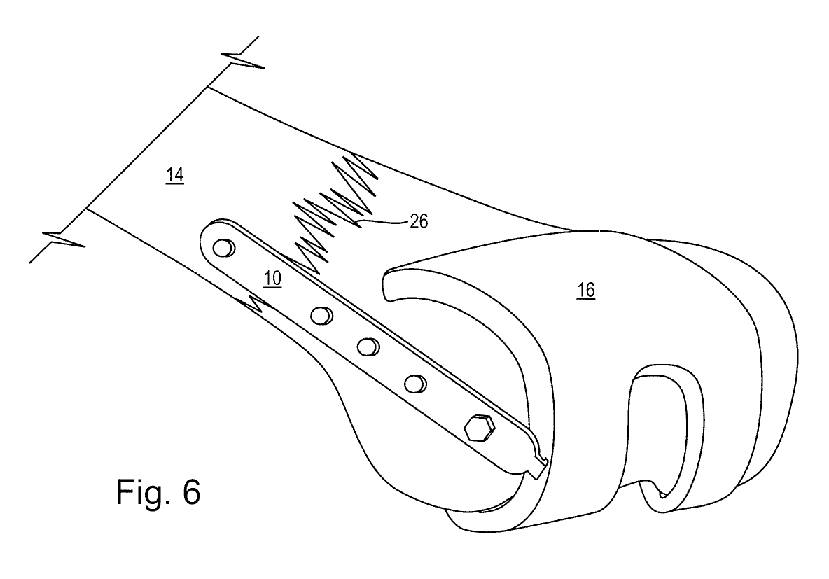

FIGS. 7 and 8 illustrate an alternative embodiment of the invention, wherein plates 10 and 12 are replaced by an intramedullary rod 100, extending through the interior of the fractured femur. Rod 100, which is adapted from known surgical rods of this type, comprises an elongate member configured for insertion within the medulla of the femur. The rod 100 is fixedly retained by an anchoring means engaged to the femoral prosthesis. According to this embodiment, a rigid anchor is provided which consists of opposing engagement members 102 for engaging the prosthesis. The engagement members comprise metal plates 104 having inwardly-turned tabs 106 configured to engage slots 34 within the implant, in the same fashion as in the embodiment of FIGS. 1-6. However, in comparison with the first embodiment described herein, these engagement members are relatively short, and are not attached directly to the femur. The respective engagement members are rigidly joined together in a compressive mode by a rigid link 50, similar to the link of the first embodiment hereof. For example, the link may comprise a threaded shaft 52, extending through an opening formed within the patient's femur bone at a location where the bone is within the femoral component of the artificial knee. Shaft 52 is retained by a threaded compression nut 54. According to this version, the intramedullary rod 100 includes an internally threaded transverse bore 110 at its distal end, through which shaft 52 may be inserted. Shaft 52 is provided with external screw threads 112 which mate with the internal threads 114 of the rod 10, so as to precisely position and fixedly retain the rod in place on the transverse shaft 110. Following joining together of the rod and shaft, the compression nut 54 is threaded onto the shaft 52, in order to apply compressive force between the engagement members 104, thereby forming a rigid structure between the transverse shaft 52 and the longitudinal intramedullary rod 100. It will be seen that the respective engagement members may comprise tabs 32 as described above in connection with the first embodiment, or alternatively the hollow cylindrical member 40 of the type shown in FIG. 9.

The embodiments described above comprise conventional surgical grade materials, such as stainless steel. It will be seen that components with metal-to-metal contact, for example between the medial and lateral plates and the knee prosthetic, must be carefully selected to minimize any possibility of bi-metallic corrosive effects.

A brief description of the surgical technique to use the device is as follows:
(1) Use a standard medial parapatellar exposure to the knee that was already used for the previous knee replacement.
(2) Slide the lateral femoral condylar locking plate 10 submuscularly proximally alongside the femur.
(3) Place the precontoured medial low profile plate 12 on the condyle and into the medial slot 34 of the femoral prosthesis.
(4) Reduce the tabs 32 of the lateral and medial plates 10 and 12 into the slots 34 of the femoral prosthesis 16 and hold in place with the plate link clamp device 60.
(5) Insert the distal fixed angled compression screw 50 and compression nut 52 to link the plates 10, 12 across the condyles in a more permanent fashion.
(6) Percutaneous K-wires can be inserted into the proximal femur through the plate holes 22 to align the limb and determine the correct length and rotation.
(7) Fill in the remaining screws 24 into the lateral plate using standard or fixed angle locking screws.

An alternative technique for linking the two plates to the femoral prosthesis with an outrigger device 120 that can be used for targeting screw placement across the plates is also shown in FIG. 4.

Another alternative technique for linking an intramedullary rod device to the femoral prosthesis by using a special locking screw with threaded ends that are linked to the slots in the femoral prosthesis on either side by an ‘L’ or curved metal plate. The metal piece fits over a screw which is locked and compressed into place with a threaded nut medially and laterally as shown in FIG. 5.

Although the present invention has been described by reference to the detailed embodiments described above, persons skilled in the art will recognize that the invention is not limited to such detailed embodiments nor the particular details of the invention described herein. Rather, the invention comprises such variations, differences, departures and equivalents of the elements described and characterized herein as would be recognized by persons skilled in the art. The full scope of the invention may be derived from the specification as a whole, including the description and claims, and further including reasonable equivalents to elements described therein.

1. An apparatus for attachment to a patient's femur to treat a fracture in the distal region of the femur, in a patient with an artificial knee joint comprising a femoral component, said apparatus comprising:
a first plate for attaching to the femur over the location of the fracture, said plate having proximal and distal ends;
first coupler at said distal end of said first plate to rigidly couple said first plate to said femoral component of the artificial knee joint;
a second plate for attachment to the femur, at a location opposed to said first plate, said second plate having proximal and distal ends;
second coupler at said distal end of said second plate to rigidly couple said second plate to said femoral component at a location spaced apart from said first coupling means; and
a transverse link for connecting said first and second plates together in a spaced apart relationship to form a rigid linkage between said plates.

2. An apparatus as defined in claim 1, further comprising a fastener to directly fasten said first and second plates to said femur.

3. An apparatus as defined in claim 1, wherein said first and second couplers each comprise a tab configured to engage a slot within said femoral component.

4. An apparatus as defined in claim 1 wherein said first and second couplers each comprise a concave member configured to engage an edge of said femoral component.

5. An apparatus as defined in claim 1 wherein said transverse link applies a compressive force urging said plates together.

6. An apparatus as defined in claim 1, wherein said transverse link comprises a fixed angle threaded compression bolt configured to extend through opposed openings within said plates and a removable threaded nut configured to mate with said bolt, and wherein at least one of said openings in said plates is threaded to mate with said bolt to retain said bolt at a predetermined angle.

7. An apparatus as defined in claim 6 wherein said predetermined angle places said femoral component at about a 5 degree valgus angle in the coronal plane and parallel to the femur in the sagittal plane.

8. An apparatus as defined in claim 1 wherein said transverse link comprises an elongate blade plate, and at least one locking member for clamping or engaging said blade plate to said plates.

9. An apparatus as defined in claim 1, wherein said first plate is configured to attach to the lateral side of said femur, and said second plate is configured to attach to the medial side of said femur, said second plate having a length selected to minimize disruption to the blood supply of the patient.

10. An apparatus as defined in claim 1, wherein said first plate is longer in length than said second plate.

11. An apparatus as defined in claim 1, further comprising a removable coupler for temporarily retaining said first and second plates in position on said femur, prior to installation of said transverse link.

12. An apparatus as defined in claim 11, wherein said removable coupler comprises a member adjustable in length, said member being configured to extend around said femur at a location adjacent to said femoral implant, and having opposing means to engage said plates at either end of said coupler.

13-20. (canceled)

21. A method of treating a distal femoral fracture, in a patient with an artificial knee joint, comprising the steps of:
(a) providing an apparatus for attachment to a patient's femur to treat a distal femoral fracture, in a patient with an artificial knee joint, comprising:
a first elongate plate for attaching to the femur over the fracture, said plate having proximal and distal ends, said distal end comprising a first coupler to rigidly couple to the femoral component of the artificial knee joint;
a second plate for attachment to the femur, at a location opposed to said first plate, said second plate having proximal and distal ends, and a second coupler at said distal end to rigidly couple to said femoral component; and
a transverse link for connecting said first and second plates to form a rigid linkage between said plates;
(b) attaching the first and second elongate plates to the opposing lateral and medial sides of the patient's femur:
(c) fastening said first and second couplers to opposing lateral and medial sides of the femoral component of the patient's artificial knee joint; and
(d) connecting said first and second plates together with said transverse link, thereby forming a rigid linkage between said first and second plates.

22. A method as defined in claim 21, wherein said step of connecting said first and second plates with said transverse link comprises fastening said link to said first and second plates, and urging said plates together to apply a compressive force therebetween to engage said femoral component.

23. A method as defined in claim 21, wherein said first and second plates are attached to said femoral component by means of a tab extending from each of said plates, engaged within opposing slots within said femoral component.

24. A method as defined in claim 21 comprising the further step of provisionally engaging said first and second plates together with a removable coupler configured to temporarily retain said first and second plates in position on said femur.

25. A method as defined in claim 24, wherein said removable coupler comprises a member adjustable in length, configured to extend around said femur at a location adjacent to said femoral implant, said method comprising the further step of adjusting said coupler to retain said first and second plates in position against said femur.

26. A method as defined in claim 21, comprising the further step of uncoupling said plates from said femoral component, after the fracture has healed, by cutting said transverse link, and optionally removing said plates.
